(19) United States
(12) Patent Application Publication
(10) Pub. No.: US 2020/0182862 A1
(43) Pub. Date: Jun. 11, 2020

(54) METHOD AND APPARATUS FOR SINGLE TUBE BLOOD DONOR SCREENING

(71) Applicant: DRK-BLUTSPENDEDIENST BADEN-WÜRTTEMBERG-HESSEN GGMBH, Frankfurt am main (DE)

(72) Inventors: MICHAEL SCHMIDT, BAD HOMBURG (DE); WALID SIREIS, KASSEL (DE); ERHARD SEIFRIED, FRANKFURT AM MAIN (DE)

(21) Appl. No.: 16/622,069

(22) PCT Filed: Jun. 15, 2018
(86) PCT No.: PCT/EP2018/066004

§ 371 Date: Dec. 12, 2019

(30) Foreign Application Priority Data
Jun. 16, 2017 (EP) 17176332.9

(51) Int. Cl. G01N 33/53; G01N 33/52; G01N 33/80

(52) U.S. Cl. CPC G01N 33/5306; G01N 33/80; G01N 33/52

(57) ABSTRACT

The present invention relates to a method for the pre-analytical treatment of a blood sample to be analyzed, comprising a suitable sample matrix for said sample and suitably centrifuging said sample. The present invention further relates to an apparatus, characterized in that it comprises suitable components for performing the method according to the present invention. The present invention further relates to the use of said apparatus according to the present invention for the automated pre-analytical treatment of a blood sample to be analyzed according to the present invention.

### DESCRIPTION OF THE INVENTION

The present invention relates to a method for the pre-analytical treatment of a blood sample to be analyzed, comprising a suitable sample matrix for said sample and suitably centrifuging said sample. The present invention further relates to an apparatus, characterized in that it comprises suitable components for performing the method according to the present invention. The present invention further relates to the use of said apparatus according to the present invention for the automated pre-analytical treatment of a blood sample to be analyzed according to the present invention.

### BACKGROUND OF THE INVENTION

Human blood is a highly valuable and hitherto indispensable raw material in medicine, which nowadays is used for extracting or manufacturing a large number of components and products.

A major step in transfusion medicine was the description of the ABO blood groups by Karl Landsteiner at the beginning of the 20^{th} century (Schwarz H P, Dorner F: Karl Landsteiner and his major contributions to haematology. Br J Haematol 2003; 121:556-565). All blood donations as well as all patients must be screened for the blood groups to enable the correct allocation. At that time medical laboratories were characterized by a high level of expertise of the employees, a high number of employees and a low number of investigated parameters.

The development of transfusion medicine as well as laboratory medicine goes on rapidly. In addition to blood grouping investigations it was reported that infections like syphilis or hepatitis B virus infections were transmitted by blood transfusions (Busch M P: HIV, HBV and HCV: new developments related to transfusion safety. Vox Sang 2000; 78 Suppl 2:253-256; Chambers R W, Foley H T, Schmidt P J: Transmission of syphilis by fresh blood components. Transfusion 1969; 9:32-34). Therefore, more screening tests were implemented into blood donor screening. Hepatitis B virus was firstly described by Blumberg et al. in 1969 (Blumberg B S, London W T: Hepatitis B virus: pathogenesis and prevention of primary cancer of the liver. Cancer 1982; 50:2657-2665). Only five years later in 1974 the first HBsAg screening test was available (Lange W, Apodaca J, Kohler H: Antikorper gegen Hepatitis B (surface)—Antigen bei Hepatitispatienten and anderen epidemiologisch interessanten Bevolkerungsgruppen. Zentralbl Bakteriol Orig A 1975; 232:199-212). This was a milestone for blood donor screening for Transfusion Transmitted Infectious Diseases (TTID).

In the middle of the last century working in a blood bank laboratory was updated to the next level (lab 2.0). This was characterized by high throughput screening analyzers (e.g. Laperche S, Sauleda S, Piron M, et al.: Evaluation of the sensitivity and specificity performance of the Elecsys® HTLV-I/II assay in a multicenter study in Europe and Japan. J Clin Microbiol 2017). These new developed screening machines were able to analyze donor samples automatically. Technicians have to fill the analyzer with all reagents disposables and controls, have to load the sample tubes and can leave the instrument alone while it is analyzing the samples. New terms “hands on time” and “work alone time” were used to characterize the level of automation for each screening system.

At the end of the last century the HIV AIDS scandal enabled the integration of a new technology into blood donor screening by the investigation of molecular parameters by nucleic acid technologies (NAT). This promising new technology started again at laboratory level 1.0. Only a few medical laboratories were able to implement this technology in the late 90-ies into blood donor screening. For NAT it was strictly necessary to have separate rooms (pre-NAT room, NAT room and post NAT room). Staff was trained to work in a one-way-direction and it was strictly forbidden to go back to the NAT room after entry the post-NAT room on the same day. The detection of RNA and DNA viruses was a further challenge to screening tests. In the beginning sample tubes have to be open again after the reverse transcription step to change the enzyme from a reverse transcriptase into a DNA polymerase. But the technique developed rapidly and complete NAT robot systems are now available for blood donor screening.

Blood donor screening can be divided into 3 parts (pre-analytic, analytic and post-analytic features). The pre-analytic is important and crucial to achieve optimal analytic test results. The analytical part can be subdivided into four groups (nucleic acid testing (NAT), serology testing, blood grouping and clinical chemistry). Up to now all blood donor services worldwide use a separate sample tube for each subpart of the analytical measurements with different pre-analytical conditions.

Therefore, it is an object of the present invention to develop a method that provides a system and method allows for a simplified but reliable pre-analytic for blood donor screening. Other objects and advantages will become apparent to the person of skill when studying the following description.

The present invention solves this object by providing a method for the pre-analytical treatment of a blood sample to be analyzed, comprising the following steps: a) providing the sample to be analyzed in a suitable container, b) providing a suitable sample matrix for said sample, comprising an anticoagulant selected from K₂EDTA, K₃EDTA and sodium citrate in said container, c) centrifuging said sample of b) at about 2000 to 3400×g for about 2 to 10 min, preferably for about 5 min at about 2600×g; and d) subjecting said sample to analytical testing comprising at least two, at least three, preferably all, of i) serology testing, ii) clinical chemistry (CC) testing, iii) nucleic acid testing (NAT); and iv) blood typing.

In a preferred embodiment, steps b) to c) of the process can be performed repeatedly.

The present invention further solves the above object by providing an apparatus, characterized in that it comprises suitable components for performing the method according to the present invention. The present invention further solves the above object by providing the use of said apparatus according to the present invention for the automated pre-analytical treatment of a blood sample to be analyzed according to the present invention.

With the present invention, a so-called “single tube management system” (STMS) and method, all measurements for blood donor pre-screening can be achieved out of one single sample tube. Said container comprises a compatible (or “harmonized”) sample matrix (EDTA or citrate), and is subjected to compatible pre-analytical conditions (compatible time and speed of centrifugation). The thus achieved substantial reduction of sample tubes to merely only one tube per donation reduces the work load at the donation site, improves blood safety by avoiding mix-up errors at the blood donation site, and reduces the cost of blood donations.

In the context of the present invention, numerous experiments were performed in order to find a technical solution to harmonize all pre-analytical conditions for one container/tube. All experiments were done using different matrices (EDTA plasma sample tubes, citrate sample tubes, serum sample tubes) and with all kinds of relevant tests (NAT: HBV, HCV and HIV; serology: HBsAg, Anti-HBc, Anti-HCV and HIV combo; blood grouping: ABO, Rhesus, Kell; clinical chemistry: IgG and total protein).

Based on the validation data as obtained, it was surprisingly found that a) it is actually possible to achieve compatible pre-analytical parameters, and i) the centrifugation time should be at about between 2 min and about 10 min with an optimal time of about 4 to 6 minutes, and the centrifugation should be between about 2,000×g and about 4,000×g with an optimal speed of about 2,400×g to about 2,800×g, with an optimum of about 2,600×g.

Within these harmonized pre-analytical conditions the sensitivity and specificity were surprisingly equivalent to the current gold standard with different pre-analytical conditions for each diagnostic group.

In the context of the present invention, unless indicated otherwise, the term “about” shall mean to include +/−10% of the value as indicated.

Preferred is the method according to the present invention, further comprising archiving analytical material of i) and/or iii). Archiving can be done in suitable vials (preferably coded) using suitable buffers. The samples are usually stored in a freezer.

Preferred is the method according to the present invention, wherein said container is labeled with a barcode. This is important for scale-up, and routine proceedings, as well as for the automated handling of samples.

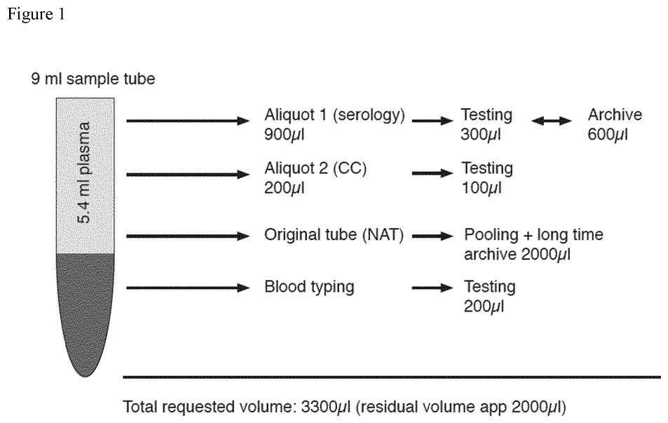

According to the present invention, the sample must have a volume that is sufficient for performing the desired tests as described herein. Preferably, the sample has a volume of about 5 to 10 ml, and preferably of about 9 ml. For the individual analytical testings, preferred volumes are selected from about 900 ul for serology, about 100 ul for CC, about 2 ml for NAT, and about 200 ul for blood typing (see also FIG. 1).

According to the present invention, any suitable vial can be used, which should be free of interfering chemicals (e.g. pyrogen-free), and stable under the desired conditions (e.g. temperature and centrifugation). Preferred is the method according to the present invention, wherein said container is a sample tube, vial, or round bottom tube.

In a preferred embodiment, the sample matrix as provided to the sample(s) to be analyzed is either provided as a solution and/or a spray dried composition (see, for example, Leathem S et al. Equivalence of spray-dried K2EDTA, spray-dried K3EDTA, and liquid K3EDTA anticoagulated blood samples for routine blood center or transfusion service testing. Immunohematology. 2003; 19(4):117-21), depending on the circumstances and the method(s) as used. Preferred according to the present invention is a final citrate concentration of about 0.005 to about 0.015 mmol/l, more preferred about 0.0109 mol/l (0.32%) or about 0.0129 mol/l (0.38%). The amount of EDTA needed to avoid blood clotting can be readily adjusted by the person of skill, and is usually between about 1.5 and about 1.8 mg per 1 ml of blood. Potassium EDTA(K2 or K3) is more preferred, rather than Sodium EDTA, because Sodium EDTA is less soluble in water. 10% solution of potassium EDTA (w/v) in distilled water is prepared as stock anticoagulant for hematological studies. To collect 1 ml blood, 10 ul of this solution is added to the collection tube.

In a preferred embodiment, the method according to the present invention is performed fully automated, without manual intervention. In a preferred embodiment, the method according to the invention represents a single homogeneous process without manual intervention.

Preferred is the method according to the present invention, wherein said blood sample to be analyzed is not a serum sample and/or does not comprise heparin. The inventive single tube management system (STMS) with compatible pre-analytical conditions is feasible for EDTA plasma samples as well as for citrate plasma samples, but it was found that it can not be used for serum samples (not feasible for blood grouping) and for heparin plasma samples (not feasible for NAT).

In another aspect of the method according to the present invention, said blood sample to be analyzed is a pooled sample, e.g. of 2 to 15 samples. Also the samples to be archived can be pooled samples. This is done in order to further streamline the process, where possible, or required. Pooling the blood samples is performed directly in containers labeled with barcodes or in the wells of plates. The method of the present invention effectively eliminates the risk of mixing up samples, which existed with the previous method that involved partial manual steps for virus enrichment. For this purpose, the pooling of blood samples occurs directly in containers labeled with barcodes or in the wells of plates.

In another aspect of the method according to the present invention, the method according to the present invention further comprises an additional centrifugation at about 2000 to 3400×g for about 15 to 25 min, preferably for about 20 min at about 2600×g, and a re-testing of serology according to the present invention, if the sample is initially reactive for said serology. That is, initially reactive samples for serology parameters could or should be re-tested in duplicate after an additional centrifugation of 20 min at 2,600×g. This method further helps to avoid unspecific serology screening results.

Another aspect of the invention then relates to an apparatus, characterized in that it comprises suitable components for performing the method according to the present invention. In a preferred embodiment, the apparatus according to the invention is suited for or suitable for generating the sample matrix, centrifugation, aliquot extraction, serology testing, clinical chemistry testing, nucleic acid extraction including, pooling, PCR preparation, blood typing, and/or raw data analysis.

The apparatus according to the invention may comprise several components: —at least one automated pipetting workstation, —at least one barcode reader, —at least one fluid processing arm, and —at least one robotic arm, and if required and preferred —at least one amplification unit and —at least one detection unit. The corresponding components are generally known to the person skilled in the art.

In a preferred embodiment of the apparatus according to the invention, all components are designed as an integrated apparatus and are located within a housing unit.

In a further preferred embodiment, the apparatus according to the invention is software-controlled. The process can be controlled with software according to the invention. The monitoring of the entire process can be achieved with software. The software monitors the entire process. In this context, the software provides worklists to the software programs of the individual sub-steps and processes, evaluates, and archives, e.g. error messages and sub-step results. The software according to the invention can preferably be programmed to integrate centrifugation, extraction, PCR preparation and real-time PCR.

Accordingly, a further aspect of the invention comprises a computer program to control and monitor the method according to the invention.

Another aspect of the invention relates to the use of an apparatus according to the present invention for the, preferably automated, pre-analytical treatment of a blood sample to be analyzed according to the present invention.

In the context of NAT, the samples are preferably analyzed for the presence of nucleic acid, preferably for the presence of the nucleic acid of a virus such as HCV, HCMV, WNV, HIV, HBV, HAV, and PB 19. Thus, viruses may be selected from the group consisting of: human immunodeficiency virus 1 and 2 (HIV-1 and HIV-2), as well as HIV-1 subgroups M, N and O, hepatitis C virus (HCV), hepatitis B virus (HBV), cytomegalia virus (CMV, HHV 5), hepatitis A virus (HAV), hepatitis E virus, parvovirus B19 (PB 19), human T cell leukemia virus I/II (HTLV I/II), West Nile virus (WNV), SARS coronavirus (SARS CoV), MERS coronavirus, dengue and other viruses, as well as EBV, HHV 8, HGV/GBVC, TTV or Chikungunya. The method also enables screening for the presence of nucleic acid of previously unknown viruses.

The NAT detection method may comprise the amplification of nucleic acids, such as PCR, TaqMan PCR, Real Time-PCR, TMA, NASBA, SDA, or LCR. A highly preferred embodiment of the method comprises nucleic acid amplification in the form of real-time PCR, which enables simultaneous online detection of the amplified nucleic acid.

In a further, particularly preferred embodiment, a sample, such as a blood donation sample, is tracked with a barcode label to the final result and is identifiable by that barcode. The automated, barcode-controlled nucleic acid extraction, amplification and detection following the concentration process rule out any mix-up of samples during the entire process.

The inventive method with compatible pre-analytical conditions is feasible for EDTA plasma samples as well as for citrate plasma samples, but it was found that it can not be used for serum samples (not feasible for blood grouping) and for heparin plasma samples (not feasible for NAT). The challenge in the context of the invention was the harmonization of the pre-analytical conditions (in particular centrifugation time and centrifugation speed) for blood grouping and for serology testing.

Therefore, a broad range of pre-analytical conditions was tested for NAT and for clinical chemistry parameters. Over all parameters the pre-analytical conditions are more limited for the diagnostic specificity compared to the diagnostic sensitivity. Therefore the harmonized optimal specification focused more on specificity data.

Hence, centrifugation time should be between 2 min and 10 min with a preferred time of 5 min and with a centrifugation speed between 2,000×g and 3,400×g with a preferred speed of 2,600×g. With new serological assays the processed testing volume for four serological tests (HBsAg, Anti-HBc, Anti-HCV and HIV combo) can be reduced to only 300 μl. As shown in FIG. 1 this enables to reduce the total number of sample tubes per donation to only one tube of 9 ml with the inventive single tube management system (STMS).

Central laboratories with approx. 6,000 blood donations per day thus can reduce the total number of sample tubes from approx. 18,000 sample tubes to 6,000 by using the method and single tube management system (STMS) of the invention. This reduces the costs on the donation side by collection of all samples, it reduces the test volume in the pre-donation bag, it reduces centrifugation time and costs for the removal of sample tubes. All sample tubes can be connected electronically at the donation side with the donation bags. The staff has to check the filling volume of the single tube to avoid underfilled sample tubes. After an automated centrifugation a first barcoded aliquot tube will be pipetted by the pre-analytic instrument for serology testing.

If clinical chemistry parameter (total protein and IgG) would be needed, a second aliquot sample tube will be prepared. The original sample tube will preferably be used for NAT and blood grouping. The STMS is an option to improve cost efficiency in automated track systems.

In summary, with EDTA plasma samples as well as with citrate plasma samples pre-analytical conditions can be harmonized, to enable STMS. Optimal pre-analytical conditions are shown with a centrifugation time of 5 minutes (range 4-6 minutes) and a centrifugation speed of 2,600×g (range 2,400×g-2,800×g). Cost efficiency can be improved by the implementation of a complete automated track system, by reducing the total number of employees, by reducing the education level of the employees and by the implementation of a single tube management system (STMS).

The present invention is now further described in the following examples with reference to the enclosed figures without being limited to the examples. For the purposes of the present invention, all references as cited herein are incorporated by reference in their entireties.

FIG. 1 shows a schematic overview of a preferred embodiment of the single tube management system according to the invention.

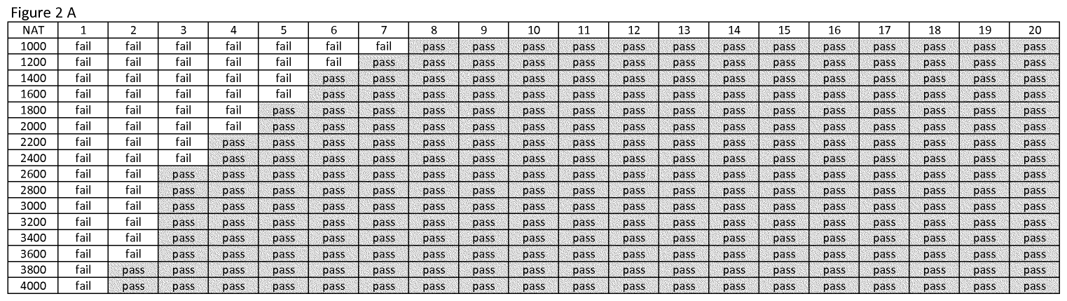

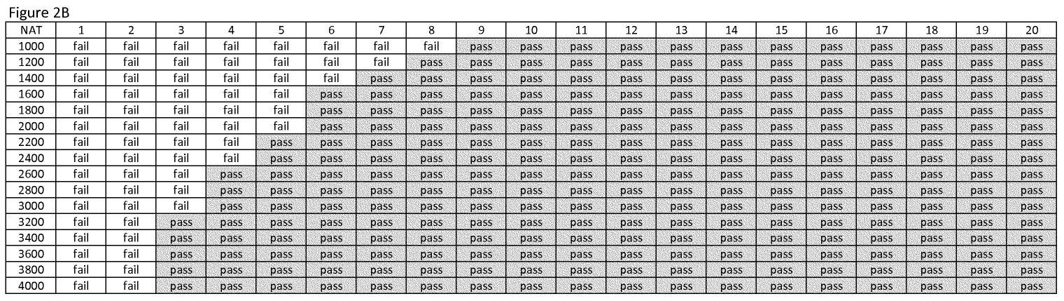

For FIGS. 2 to 21, the x-axis represents the centrifugation time (minutes, from 1 (left) to 20 (right) per column) and the y-axis the centrifugation in g (from 1000 (top) 4000 (bottom) in 200×g increments). Dark gray boxes=fail; light gray boxes=pass.

FIG. 2A shows the analysis of NAT testing for HBV for diagnostic sensitivity. FIG. 2B shows the analysis of NAT testing for HBV for diagnostic specificity.

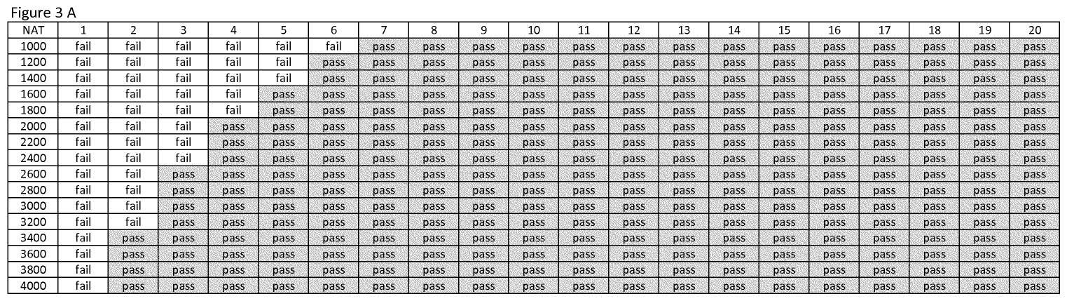

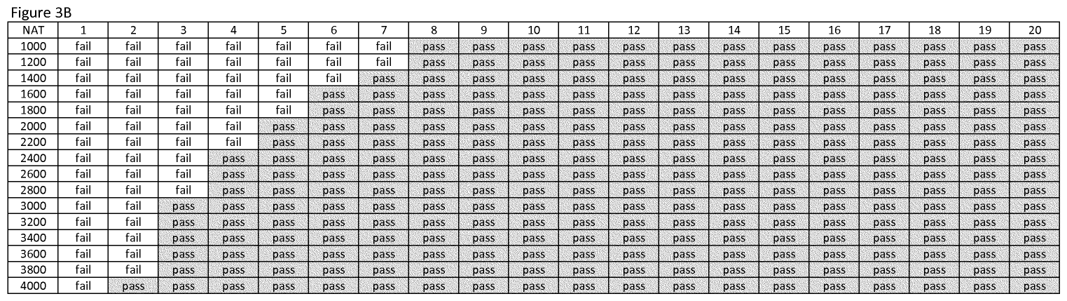

FIG. 3A shows the analysis of NAT testing for HCV for diagnostic sensitivity. FIG. 3B shows the analysis of NAT testing for HCV for diagnostic specificity.

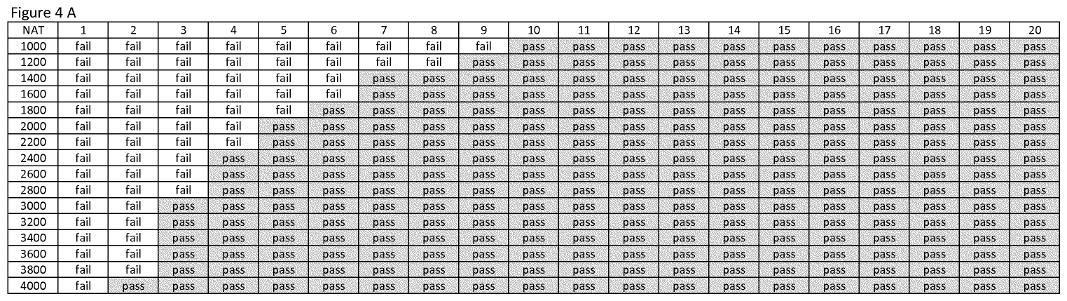

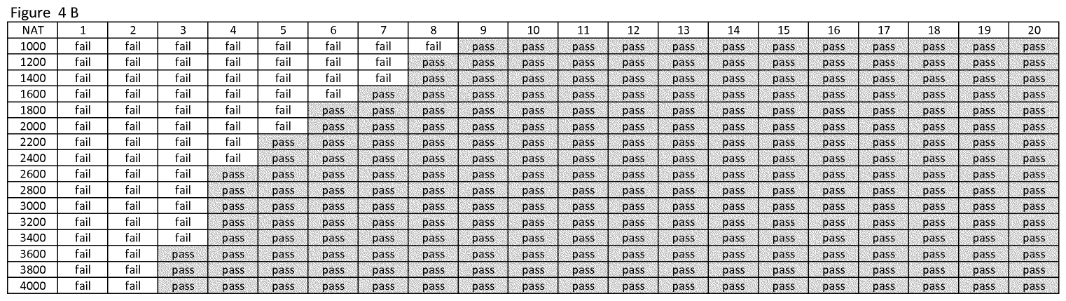

FIG. 4A shows the analysis of NAT testing for HIV for diagnostic sensitivity. FIG. 4B shows the analysis of NAT testing for HIV for diagnostic specificity.

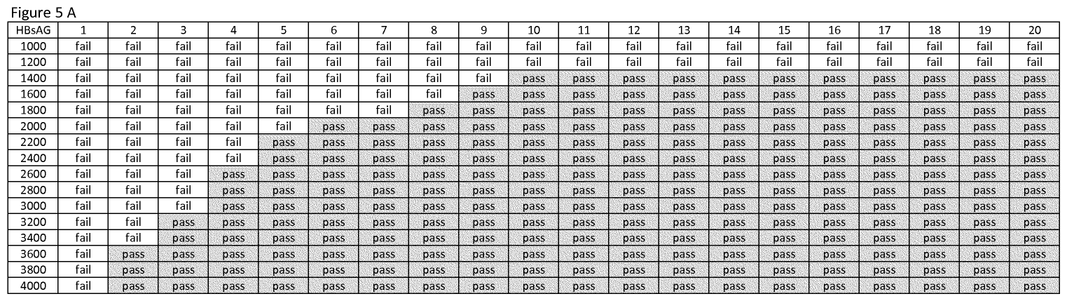

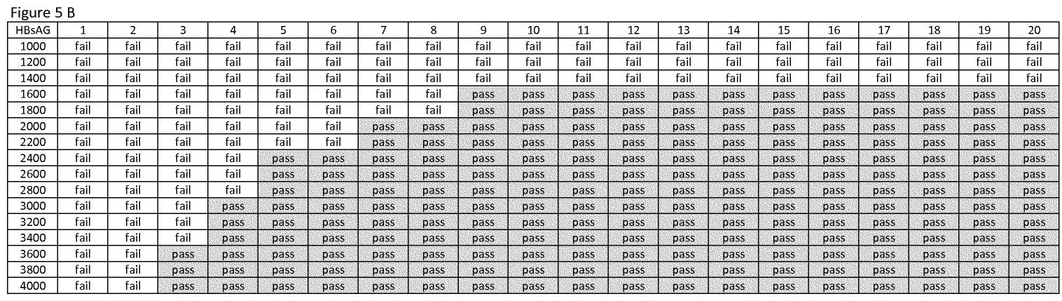

FIG. 5A shows the analysis of serology testing for HBsAg for diagnostic sensitivity. FIG. 5B shows the analysis of serology testing for HBV for diagnostic specificity.

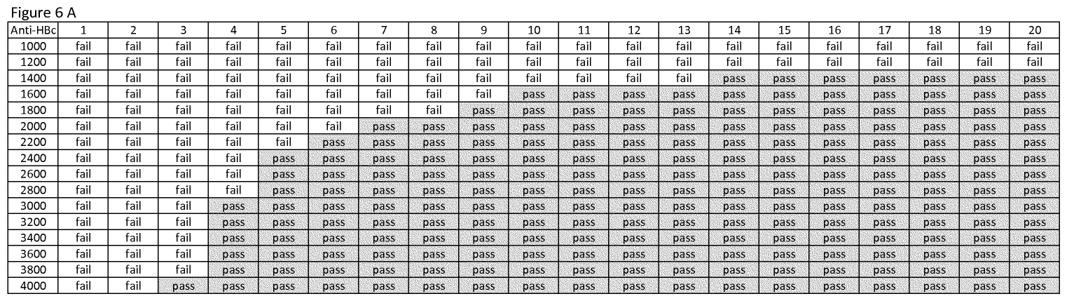

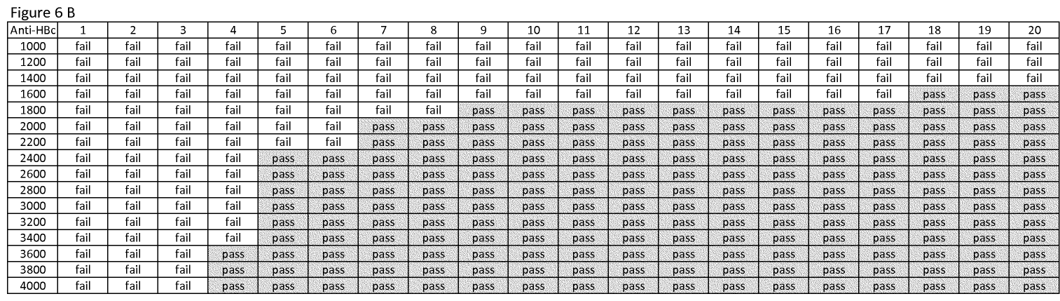

FIG. 6A shows the analysis of serology testing for anti-HBc for diagnostic sensitivity. FIG. 6B shows the analysis of serology testing for anti-HBc for diagnostic specificity.

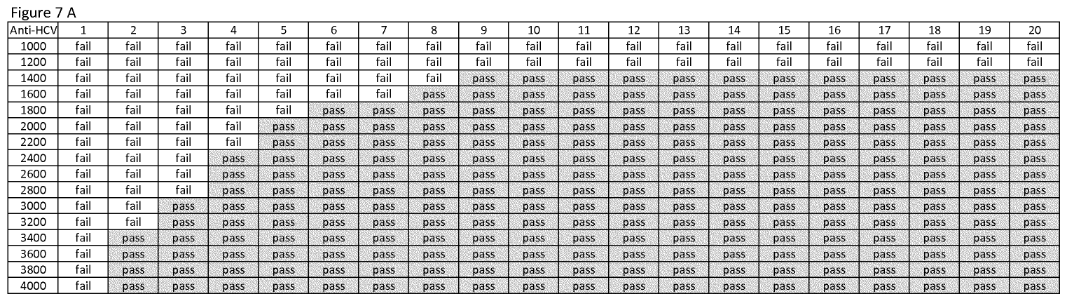

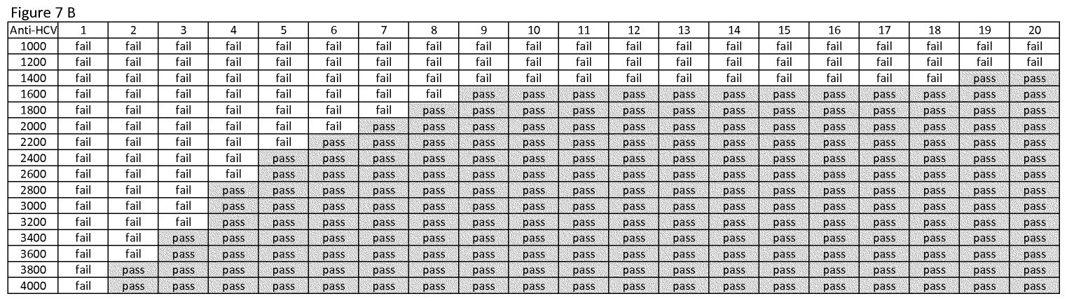

FIG. 7A shows the analysis of serology testing for anti-HCV for diagnostic sensitivity. FIG. 7B shows the analysis of serology testing for anti-HCV for diagnostic specificity.

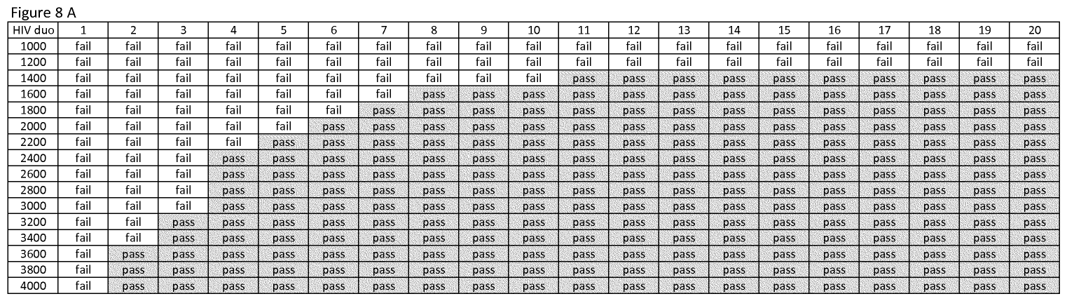

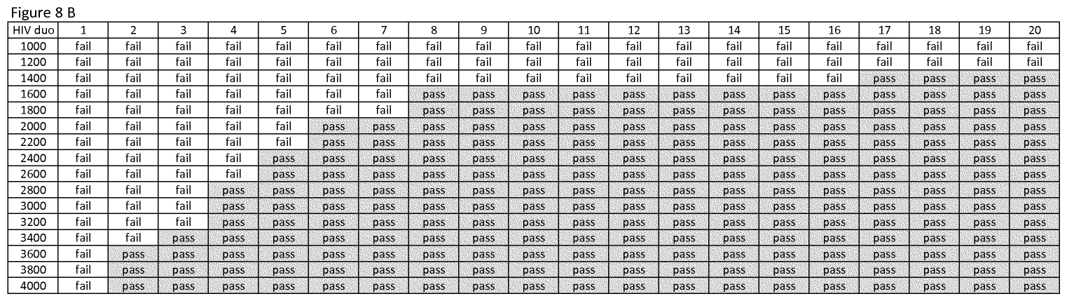

FIG. 8A shows the analysis of serology testing for HIV duo for diagnostic sensitivity. FIG. 8B shows the analysis of serology testing for HIV duo for diagnostic specificity.

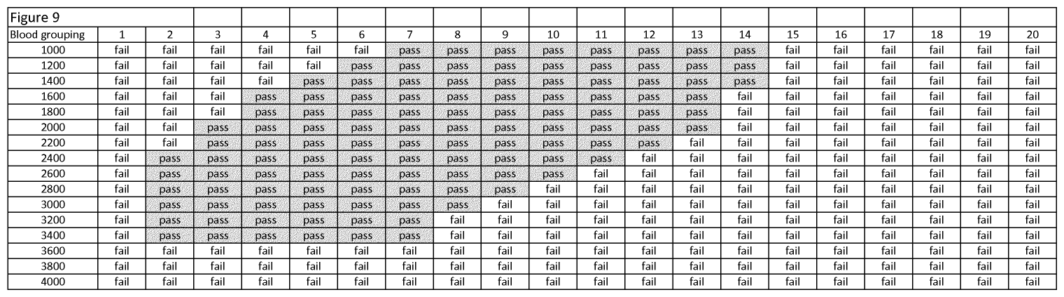

FIG. 9 shows the analysis of serology testing for blood grouping.

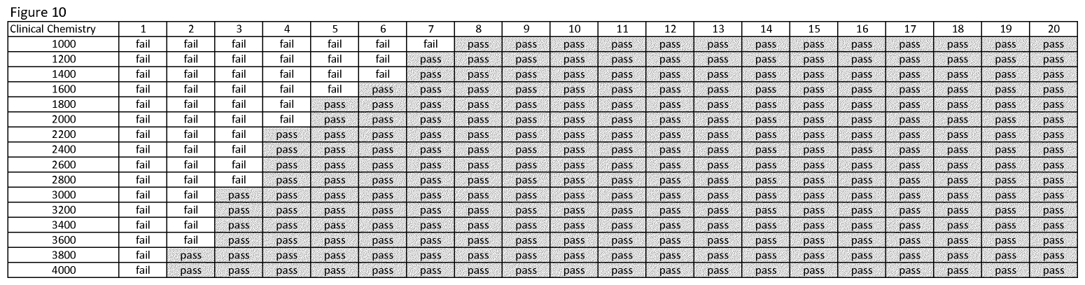

FIG. 10 shows the analysis of clinical chemistry testing for IgG.

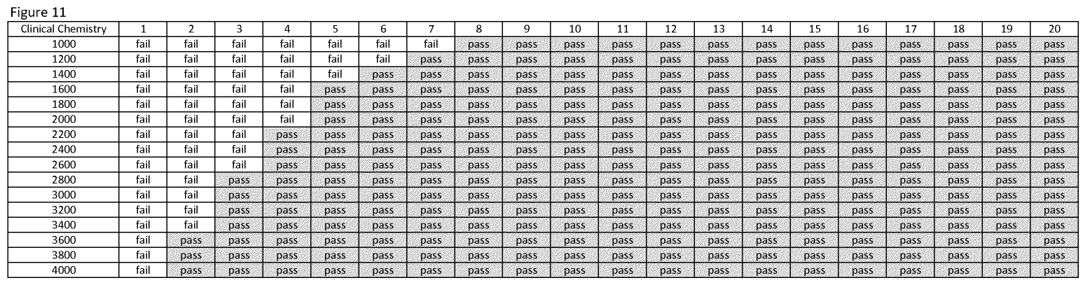

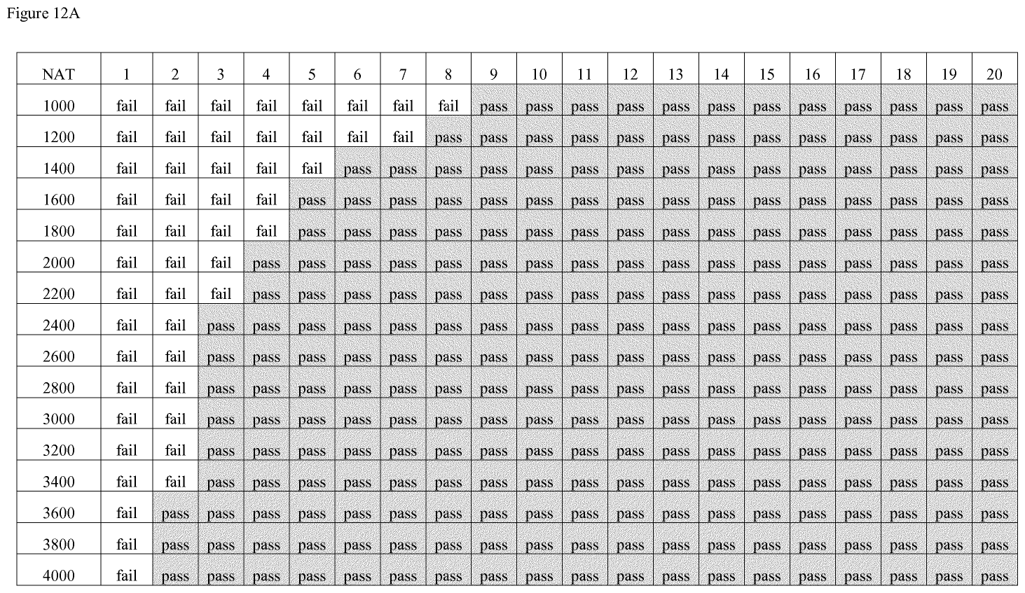

FIG. 11 shows the analysis of clinical chemistry testing for total protein.

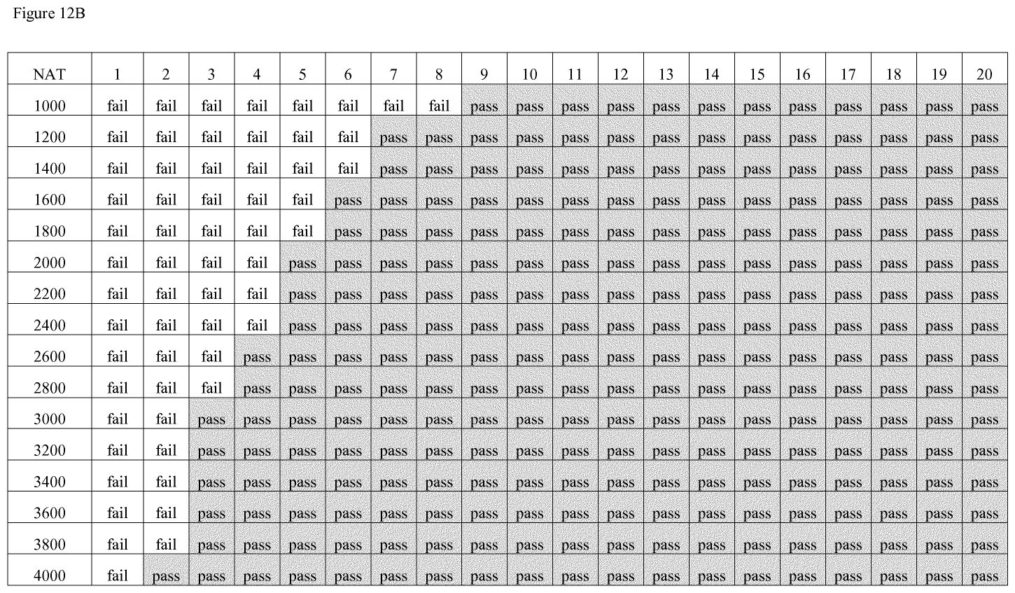

FIG. 12A shows the analysis of NAT testing for HBV for diagnostic sensitivity. FIG. 12B shows the analysis of NAT testing for HBV for diagnostic specificity.

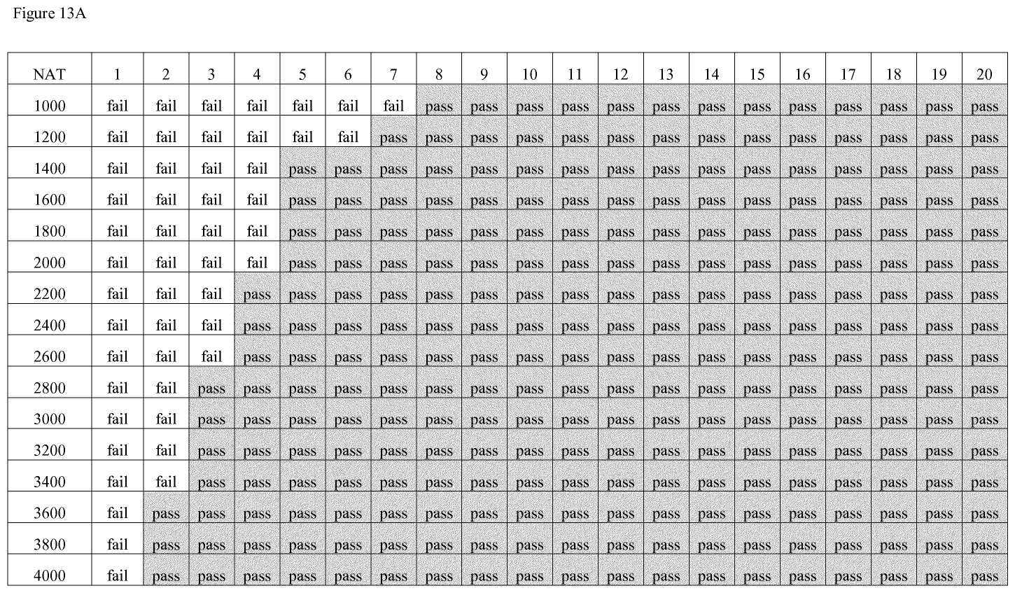

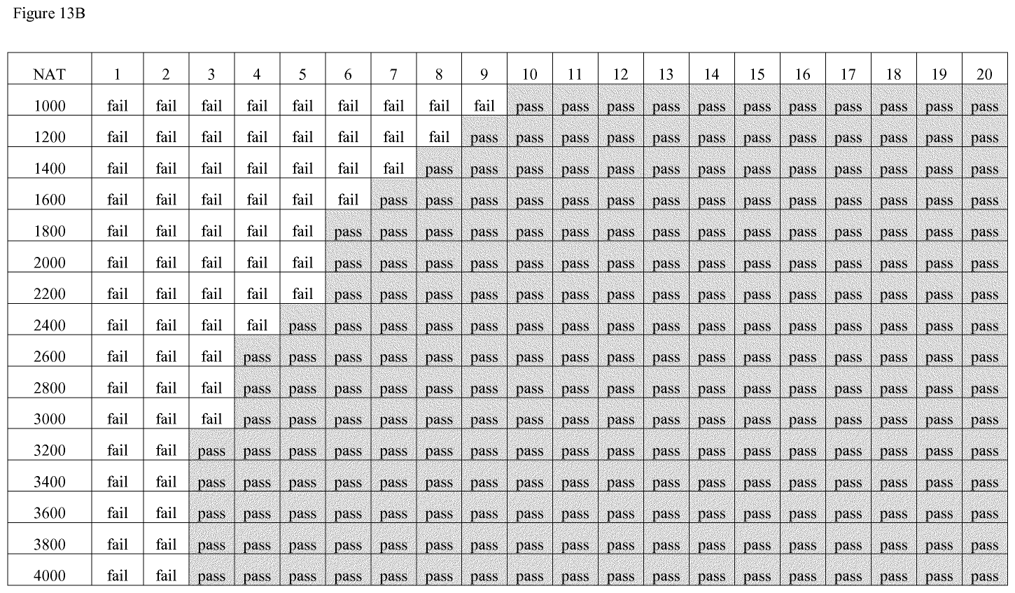

FIG. 13A shows the analysis of NAT testing for HCV for diagnostic sensitivity. FIG. 13B shows the analysis of NAT testing for HCV for diagnostic specificity.

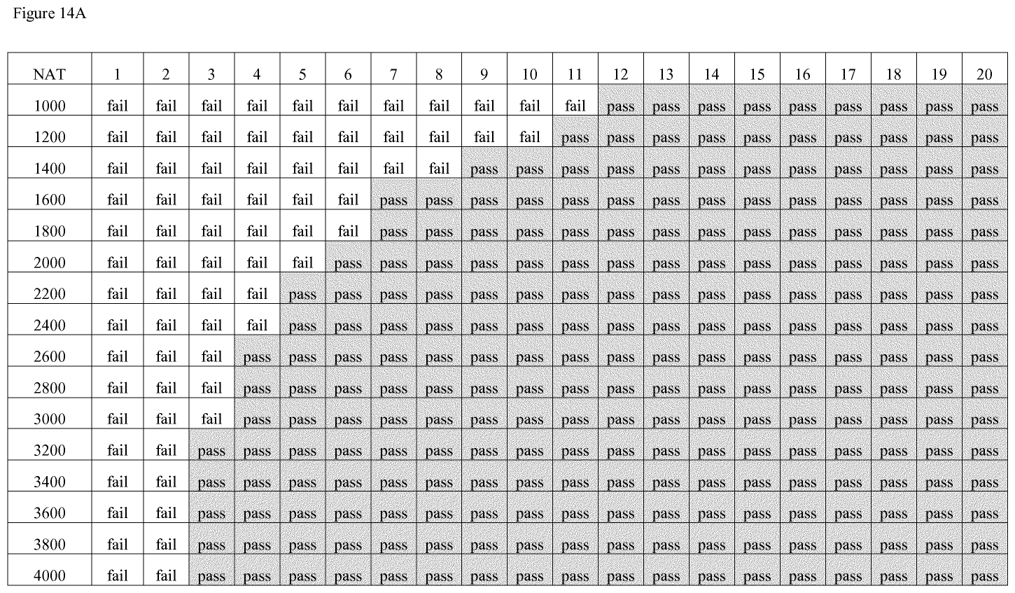

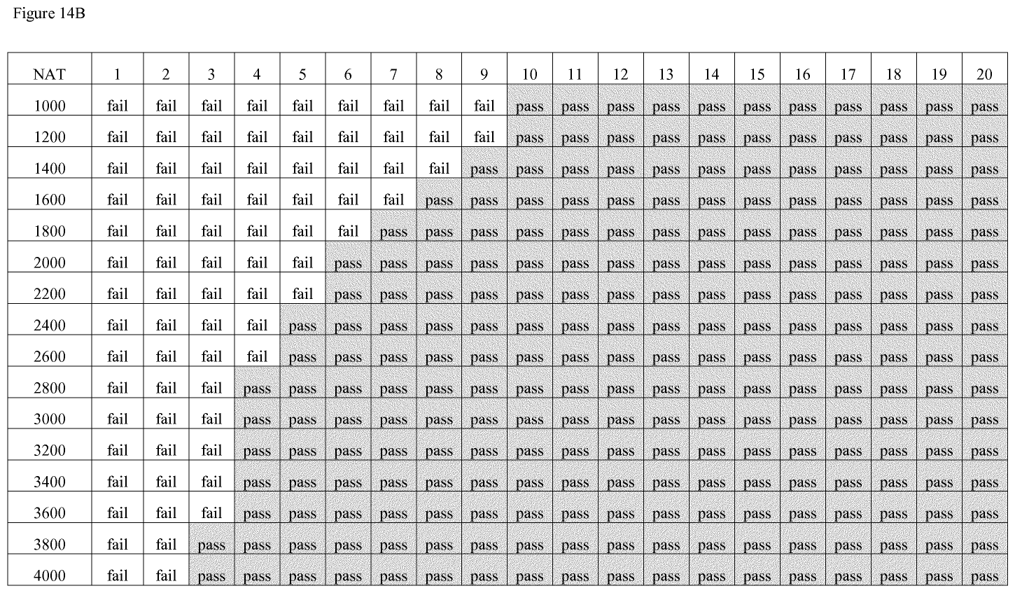

FIG. 14A shows the analysis of NAT testing for HIV for diagnostic sensitivity. FIG. 14B shows the analysis of NAT testing for HIV for diagnostic specificity.

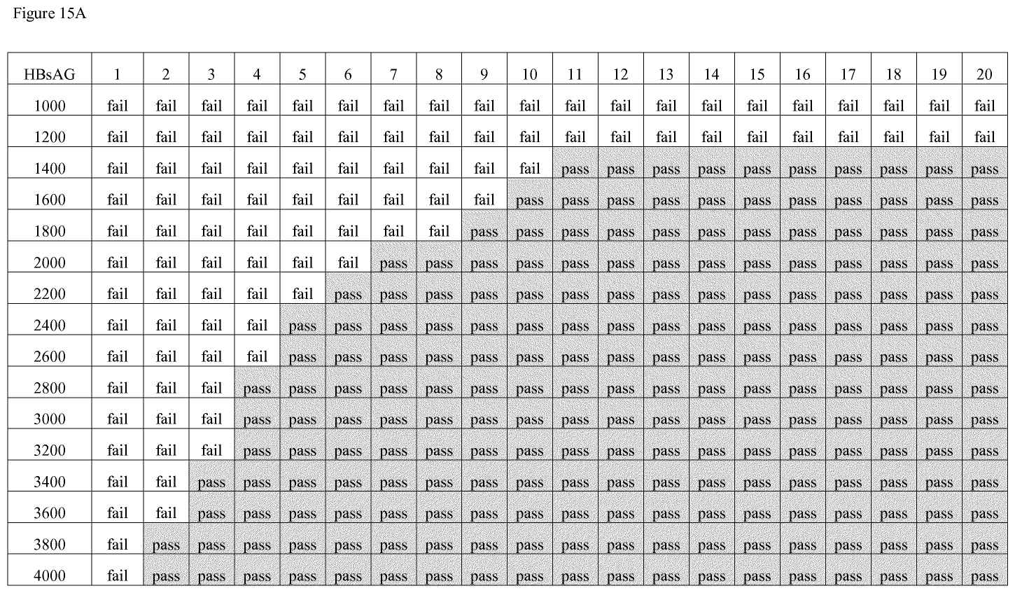

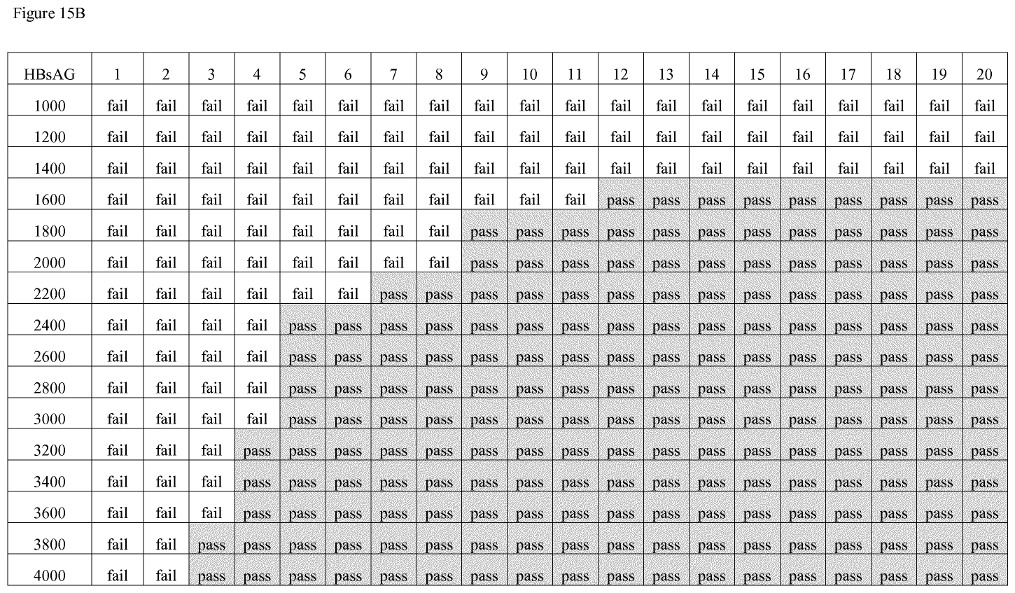

FIG. 15A shows the analysis of serology testing for HBsAg for diagnostic sensitivity. FIG. 15B shows the analysis of serology testing for HBV for diagnostic specificity.

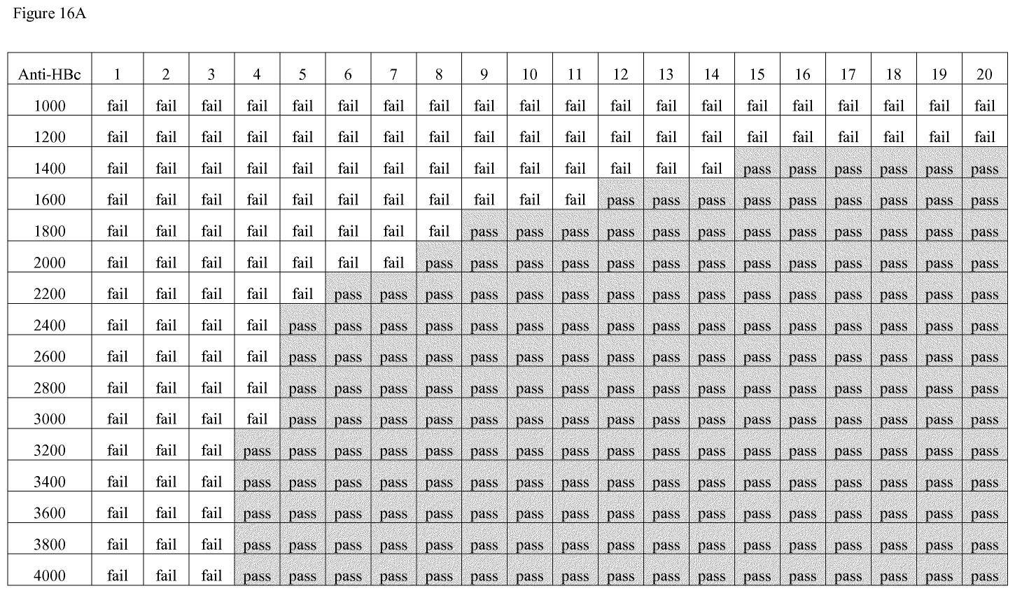

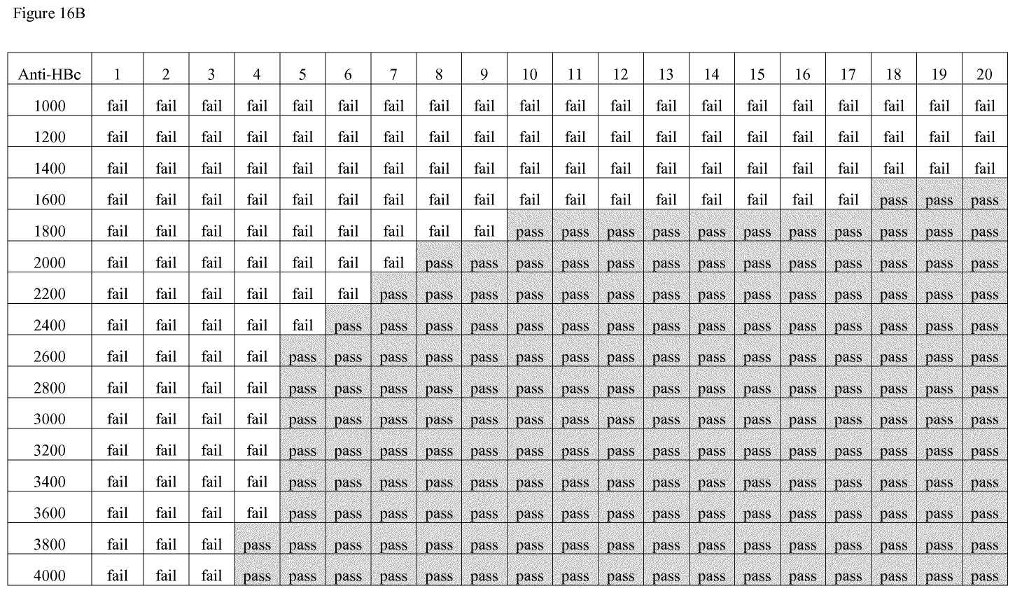

FIG. 16A shows the analysis of serology testing for anti-HBc for diagnostic sensitivity. FIG. 16B shows the analysis of serology testing for anti-HBc for diagnostic specificity.

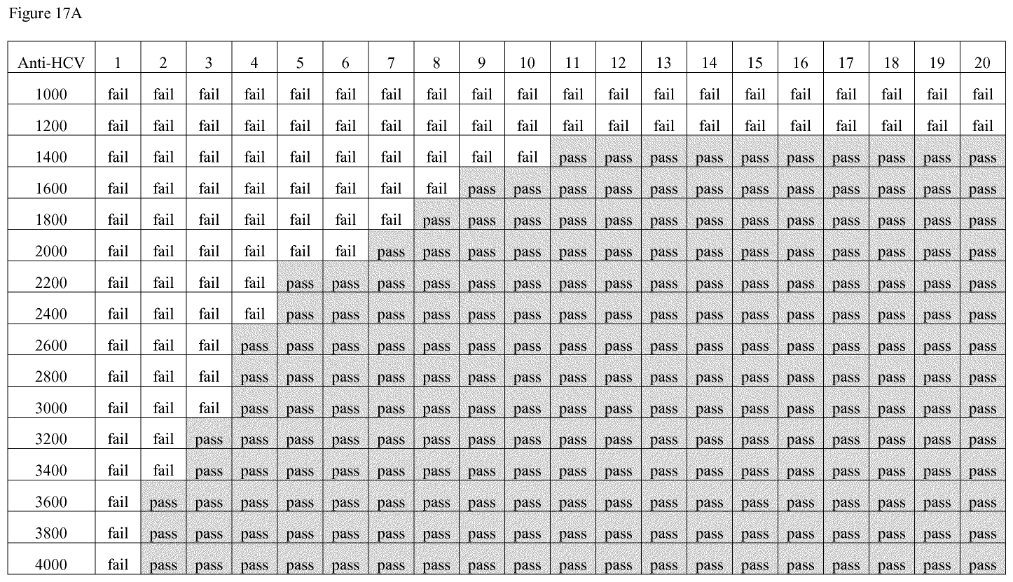

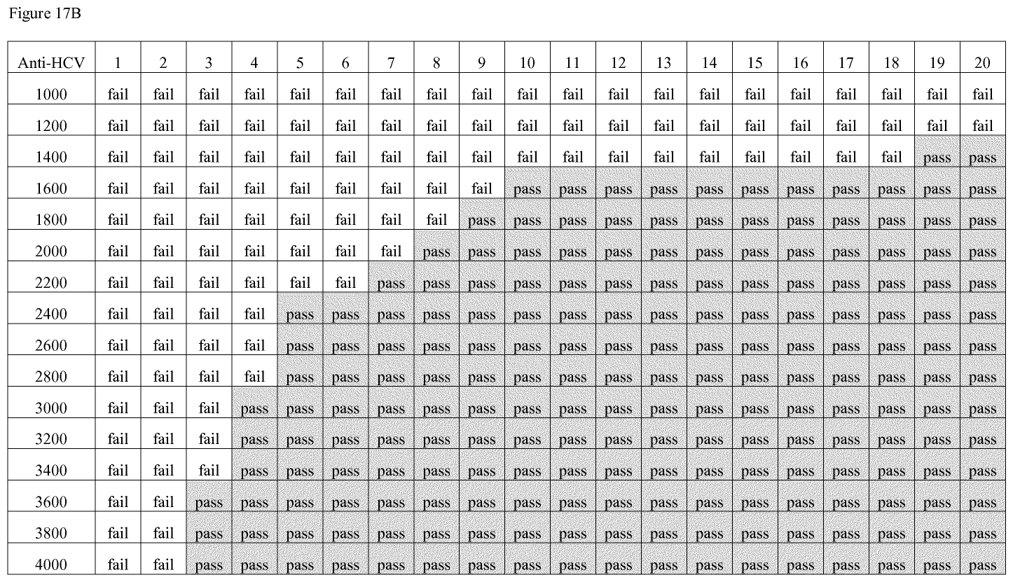

FIG. 17A shows the analysis of serology testing for anti-HCV for diagnostic sensitivity. FIG. 17B shows the analysis of serology testing for anti-HCV for diagnostic specificity.

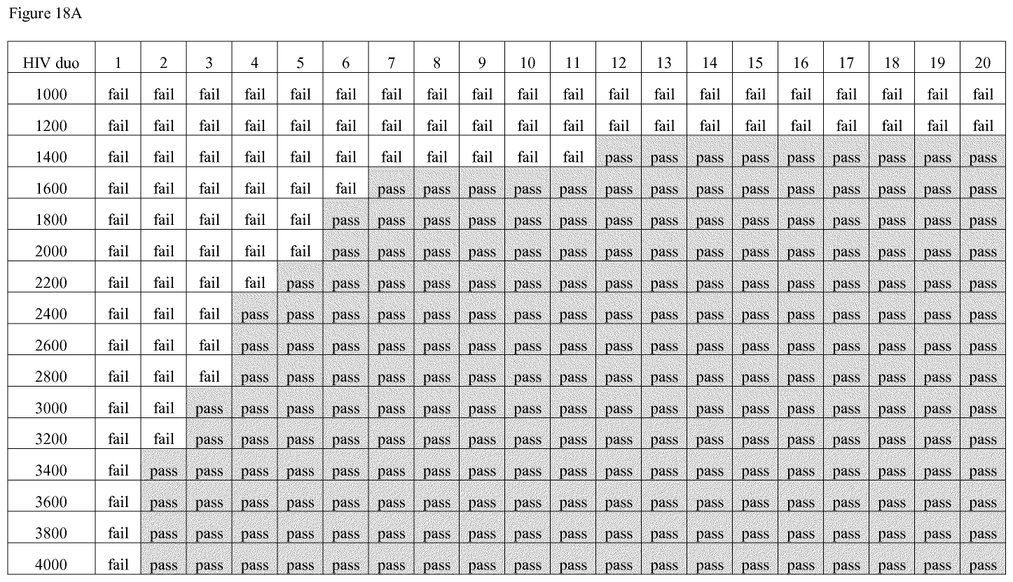

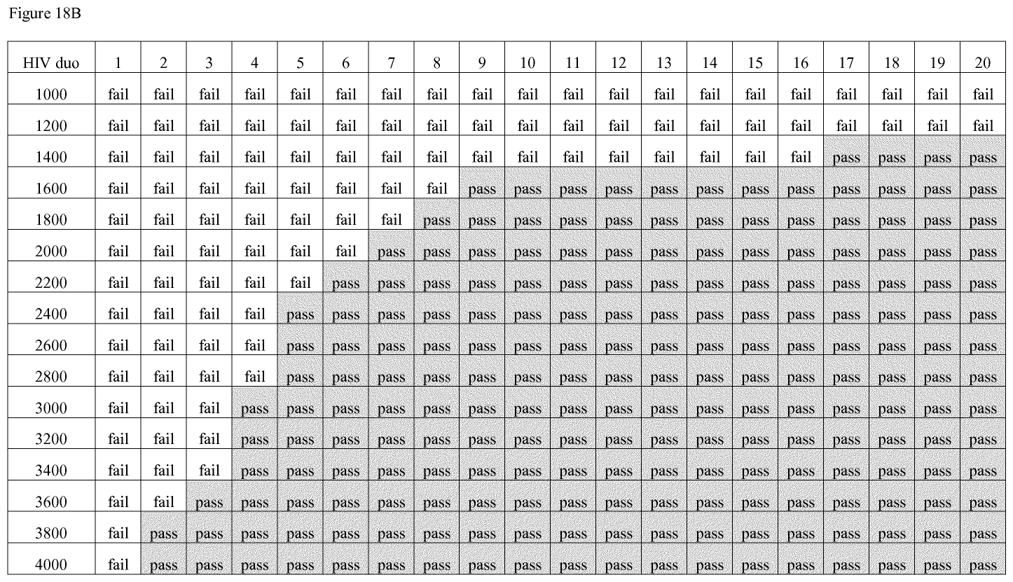

FIG. 18A shows the analysis of serology testing for HIV duo for diagnostic sensitivity. FIG. 18B shows the analysis of serology testing for HIV duo for diagnostic specificity.

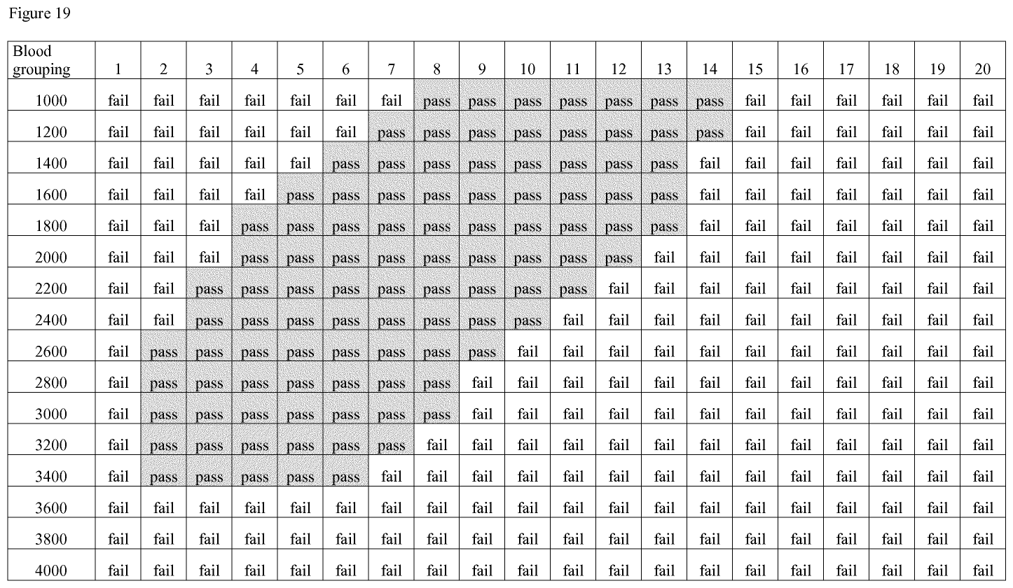

FIG. 19 shows the analysis of serology testing for blood grouping.

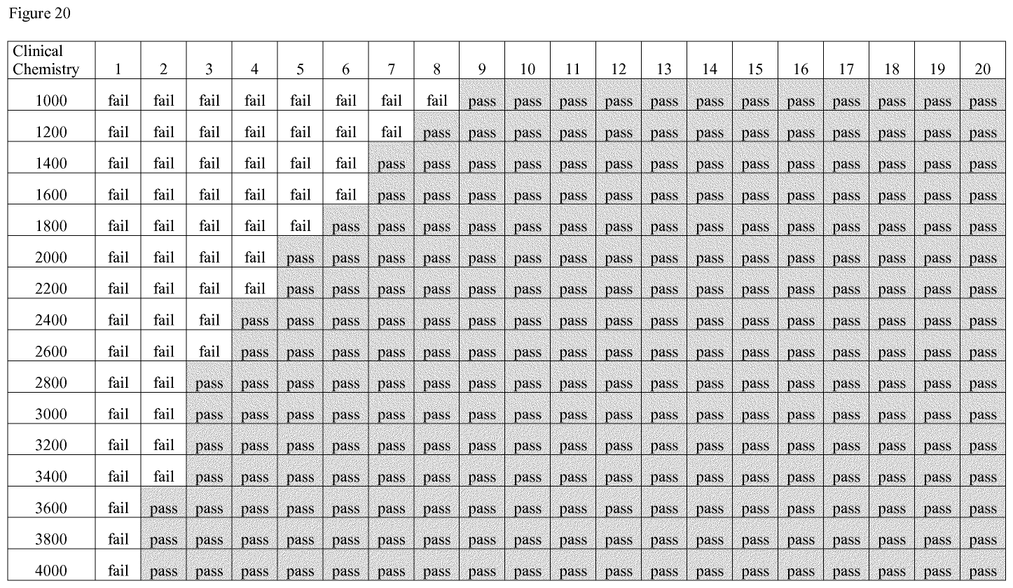

FIG. 20 shows the analysis of clinical chemistry testing for IgG.

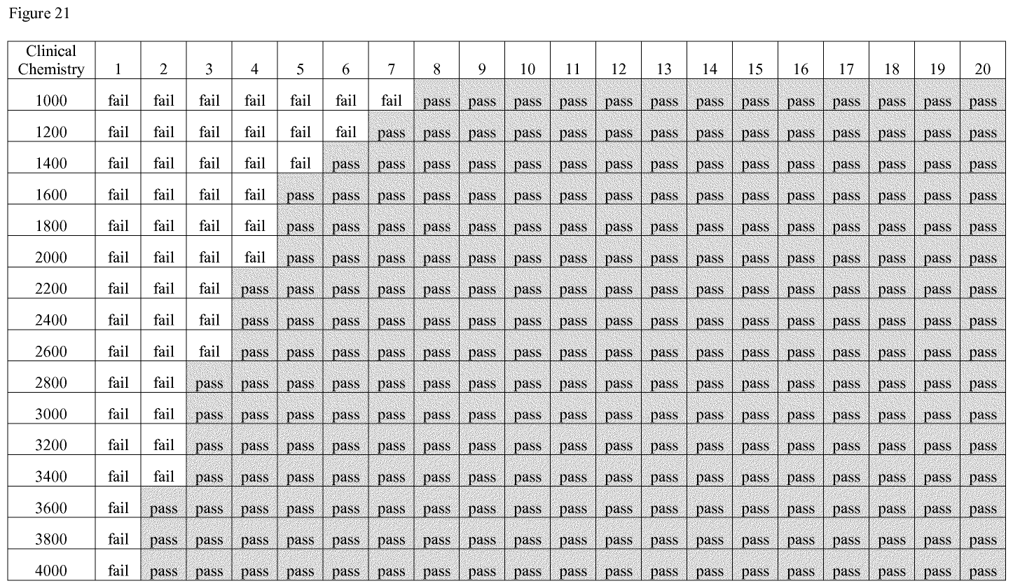

FIG. 21 shows the analysis of clinical chemistry testing for total protein.

### EXAMPLES

According to the data from an international study by the ISBT, many countries started with an implementation of NAT for HCV. After multiplex assays were available, HIV and HBV tests were added. As presented in the International Forum on NAT testing, the NAT results were 244, 680 for HIV-1, HCV and 1,728 for HBV, respectively, after screening of approx. 300 million blood donors for HIV-1 and HCV and 114 million blood donors for HBV. Current screening NAT robot systems (e.g. Roche Cobas 8800 or Grifols Panther) (Grabarczyk P, Koppelman M, Boland F, et al.: Inclusion of human immunodeficiency virus Type 2 (HIV-2) in a multiplex transcription-mediated amplification assay does not affect detection of HIV-1 and hepatitis B and C virus genotypes: a multicenter performance evaluation study. Transfusion 2015; 55:2246-2255) have implemented different chambers to separate the pre-NAT room, from the NAT room and the post-NAT room. Working with these all-in-one NAT systems needs only trained staff, input of individual samples or mini-pools, reagents and disposable. The special requirements for NAT detection were stopped. The instruments can be placed directly to other screening analyzer for serology or for blood grouping.

For all categories of blood donor screening (NAT, serology, blood grouping and clinical chemistry) tubes with different sample matrix and different pre-analytical conditions were used. Table 1 shows the current specification for sample tubes and centrifugation conditions per diagnostic group

TABLE 1
Current pre-analytical conditions

| | | Centrifugation | Centrifugation |
|---|---|---|---|
| Diagnostic group | Sample matrix | time | speed |
| NAT | EDTA, Citrate | 5 min | 2,600 + g |
| Serology | Serum | 20 min | 3,000 + g |
| Blood grouping | EDTA | 5 min | 2,600 + g |
| Clinical chemistry | EDAT, Citrate | 10 min | 3,000 + g |

The object of the present invention is to achieve a compatibility (harmonization) of the sample tube matrix and the pre-analytical conditions (in particular centrifugation time and centrifugation speed) to enable all blood donor screening measurements from one sample tube without a substantial reduction of the diagnostic sensitivity and the diagnostic specificity.

Material and Methods:

Sample Tubes

Greiner Bio One 9 ml EDTA sample tube (455036)

Greiner Bio One 9 ml Citrate sample tube (455322)

Centrifugation

All sample tubes were centrifuged using Hettich Rotixa centrifuges. Centrifugation speed is presented in speed×g (g=9.81 m/s²)

Nucleic Acid Testing (NAT)

All NAT tests were performed on the Roche Cobas 8800 instruments with the Roche MPX assay for HBV, HCV and HIV-1.

Serology Testing

All serology tests were performed on the Roche Cobas 8000 system on the 801 instrument for the parameter HBsAg, Anti-HBc, Anti-HCV and HIV duo.

Clinical Chemistry Testing

All clinical chemistry tests were performed on the Roche Cobas 8000 system on the 501 instrument for the parameter IgG and total protein.

Blood Grouping Tests

All blood grouping tests were performed on the Beckman Coulter PK7300 instrument for the parameter A, B, 0, Rhesus and Kell. The following reagents were used in order to analyze the antigens and antibodies.

TABLE 2
Blood grouping reagents

| | | | Volume | Volume | Volume |
|---|---|---|---|---|---|
| | | | concentrate | concentrate | concentrate |
| | | Manu- | to 200 ml | to 400 ml | to 600 ml |
| Reagent | Dilution | facturer | NaCl | NaCl | NaCl |
| Anti-A | 1:320 | Biorad | 625 μl | 1250 μl | 1875 μl |
| Anti-A | 1:320 | Ortho | 625 μl | 1250 μl | 1875 μl |
| Anti-B | 1:80 | Biorad | 2500 μl | 5000 μl | 7500 μl |
| Anti-B | 1:160 | Ortho | 1250 μl | 2500 μl | 3750 μl |
| Anti-D | 1:80 | Biorad | 2500 μl | 5000 μl | 7500 μl |
| mon. Clon | | | | | |
| Anti-D mon. | 1:10 | Diagast | 20.000 μl | 40.000 μl | 60.000 μl |
| Anti-D | 1:40 | Biorad | 5000 μl | 10.000 μl | 15.000 μl |
| mon. Clon | | | | | |
| Anti-C mon. | 1:160 | SD-Nostic | 1250 μl | 2500 μl | 3750 μl |
| Anti-C mon. | 1:80 | Immucor | 2500 μl | 5000 μl | 7500 μl |
| Anti-c mon. | 1:160 | Immucor | 1250 μl | 2500 μl | 3750 μl |
| Anti-c mon. | 1:80 | BAG | 2500 μl | 5000 μl | 7500 μl |
| Anti-E mon. | 1:80 | Biorad | 2500 μl | 5000 μl | 7500 μl |
| Anti-E mon. | 1:320 | Immucor | 625 μl | 1250 μl | 1875 μl |
| Anti-e mon. | 1:160 | Biorad | 1250 μl | 2500 μl | 3750 μl |
| Anti-e mon. | 1:160 | Immucor | 1250 μl | 2500 μl | 3750 μl |
| RH Kontrolle | 1:80 | Biorad | 2500 μl | 5000 μl | 7500 μl |
| Anti-Kell | 1:80 | SD-Nostic | 2500 μl | 5000 μl | 7500 μl |
| mon. | | | | | |
| Anti-Kell | 1:20 | BAG | 10.000 μl | 20.000 μl | 30.000 μl |
| mon. | | | | | |

Detection of the Diagnostic Sensitivity for NAT

WHO standards for HBV (10/264), for HCV (06/102) and for HIV-1 (10/152) were diluted to final concentrations of 10 IU/ml, 50 IU/ml and 100 IU/ml, respectively. The final virus concentration was spiked into whole blood samples. Each concentration was tested for each pre-analytical condition (matrix belong on centrifugation time and centrifugation speed) in replicates of 10. Data were analyzed by the number of positive NAT tests divided with the number of tested samples multiplied by 100. The studies on NAT were regarded as successful, if the diagnostic sensitivity was at least 90%.

Negative blood donor samples were tested for each pre-analytical condition (matrix belong on centrifugation time and centrifugation speed) in replicates of 100. Data were analyzed by the number of negative NAT tests divided with the number of tested samples multiplied by 100. The studies on NAT were regarded as successful if the diagnostic specificity was at least 95%.

Detection of the Diagnostic Sensitivity for Serology

Plasma from positive blood donors for HBsAg, anti-HBc, anti-HCV and anti-HIV-1 were diluted to final concentrations of 10 S/Co, 0.5 S/Co, 10 S/Co and 10 S/Co, respectively. The anti-HBc test was performed as a competitive test, therefore positive samples have a S/Co value below 1.0. The final virus concentration was spiked into whole blood samples. Each concentration was tested for each pre-analytical condition (matrix belong on centrifugation time and centrifugation speed) in replicates of 10. Data were analyzed by the number of positive NAT tests divided with the number of tested samples multiplied by 100. The studies on serology were regarded as successful, if the diagnostic sensitivity was at least 90%.

Detection of the Diagnostic Specificity for Serology

Negative blood donor samples were tested for each pre-analytical condition (matrix belong on centrifugation time and centrifugations speed) in replicates of 100. Data were analyzed by the number of negative NAT tests divided with the number of tested samples multiplied by 100. The studies on serology are evaluated as successful if the diagnostic specificity is at least 95%.

Detection of Blood Groups

Whole blood donor samples with blood groups listed in table 3 were divided into two parts and tested under the current routine conditions (see table 1) and under different pre-analytical conditions (matrix belong on centrifugation time and centrifugations speed) in replicates of 5. Pre-analytical conditions were accepted, if the blood grouping test results were identical to the routine standard.

TABLE 3
Blood groups as investigated

| AB0 | Rhesus | Kell (K+) | Replicates |
|---|---|---|---|
| A | Neg | Neg | 5 |
| A | Pos | Neg | 5 |
| A | Neg | Pos | 5 |
| A | Pos | Pos | 5 |
| B | Neg | Neg | 5 |
| B | Pos | Neg | 5 |
| B | Neg | Pos | 5 |
| B | Pos | Pos | 5 |
| AB | Neg | Neg | 5 |
| AB | Pos | Neg | 5 |
| AB | Neg | Pos | 5 |
| AB | Pos | Pos | 5 |
| 0 | Neg | Neg | 5 |
| 0 | Pos | Neg | 5 |
| 0 | Neg | Pos | 5 |
| 0 | Pos | Pos | 5 |

The studies on blood grouping were regarded as successful if the diagnostic sensitivity comparable with the test results under current routine conditions.

Detection of IgG and Total Protein

Whole blood donor samples were divided into two parts and tested under the current routine conditions (see table 1) and under different pre-analytical conditions (matrix belong on centrifugation time and centrifugation speed) in replicates of 10. Pre-analytical conditions could be accepted, if the quantitative data between the routine standard conditions and the new pre-analytical conditions were not significant different (p-value >0.05) by the T-Test.

Centrifugation Time:

The different centrifugation times were examined from 1 minute to 20 minutes at intervals of 1 minute each.

Centrifugation Speed:

The different centrifugation speeds were examined from 1,000×g to 4,000×g at intervals of 200×g each.

Results:

1. Matrix EDTA Plasma Samples, NAT Detection

Diagnostic Sensitivity HBV

FIG. 2A shows the analysis of NAT testing for HBV for diagnostic sensitivity. The x-axis represents the centrifugation time (minutes) and the y-axis the centrifugation speed. The pre-analytical conditions were evaluated as successful (pass) if at least 9/10 (90%) tests achieved a positive test result. The pre-analytical conditions were regarded as not successful if less than 9/10 (90%) tests achieved a positive test result.

Diagnostic Specificity HBV

FIG. 2B shows the analysis of NAT testing for HBV for diagnostic specificity. The x-axis represents the centrifugation time (minutes) and the y-axis the centrifugation speed. The pre-analytical conditions were evaluated as successful (pass) if at least 95/100 (95%) tests achieved a positive test result. The pre-analytical conditions were regarded as not successful if less than 95/100 (95%) tests achieved a positive test result.

Diagnostic Sensitivity HCV

FIG. 3A shows the analysis of NAT testing for HCV for diagnostic sensitivity. The x-axis represents the centrifugation time (minutes) and the y-axis the centrifugation speed. The pre-analytical conditions were evaluated as successful (pass) if at least 9/10 (90%) tests achieved a positive test result. The pre-analytical conditions were regarded as not successful if less than 9/10 (90%) tests achieved a positive test result.

Diagnostic Specificity HCV

FIG. 3B shows the analysis of NAT testing for HCV for diagnostic specificity. The x-axis represents the centrifugation time (minutes) and the y-axis the centrifugation speed. The pre-analytical conditions were evaluated as successful (pass) if at least 95/100 (95%) tests achieved a positive test result. The pre-analytical conditions were regarded as not successful if less than 95/100 (95%) tests achieved a positive test result.

Diagnostic Sensitivity HIV

FIG. 4A shows the analysis of NAT testing for HIV for diagnostic sensitivity. The x-axis represents the centrifugation time (minutes) and the y-axis the centrifugation speed. The pre-analytical conditions were evaluated as successful (pass) if at least 9/10 (90%) tests achieved a positive test result. The pre-analytical conditions were regarded as not successful if less than 9/10 (90%) tests achieved a positive test result.

Diagnostic Specificity HIV

FIG. 4B shows the analysis of NAT testing for HIV for diagnostic specificity. The x-axis represents the centrifugation time (minutes) and the y-axis the centrifugation speed. The pre-analytical conditions were evaluated as successful (pass) if at least 95/100 (95%) tests achieved a positive test result. The pre-analytical conditions were regarded as not successful if less than 95/100 (95%) tests achieved a positive test result.

2. Matrix EDTA Plasma Samples, Serology Detection

Diagnostic Sensitivity HBsAG

FIG. 5A shows the analysis of serology testing for HBsAg for diagnostic sensitivity. The x-axis represents the centrifugation time (minutes) and the y-axis the centrifugation speed. The pre-analytical conditions were evaluated as successful (pass) if at least 9/10 (90%) tests achieved a positive test result. The pre-analytical conditions were regarded as not successful if less than 9/10 (90%) tests achieved a positive test result.

Diagnostic Specificity HBsAg

FIG. 5B shows the analysis of serology testing for HBV for diagnostic specificity. The x-axis represents the centrifugation time (minutes) and the y-axis the centrifugation speed. The pre-analytical conditions were evaluated as successful (pass) if at least 95/100 (95%) tests achieved a positive test result. The pre-analytical conditions were regarded as not successful if less than 95/100 (95%) tests achieved a positive test result.

Diagnostic Sensitivity Anti-HBc

FIG. 6A shows the analysis of serology testing for anti-HBc for diagnostic sensitivity. The x-axis represents the centrifugation time (minutes) and the y-axis the centrifugation speed. The pre-analytical conditions were evaluated as successful (pass) if at least 9/10 (90%) tests achieved a positive test result. The pre-analytical conditions were regarded as not successful if less than 9/10 (90%) tests achieved a positive test result.

Diagnostic Specificity Anti-HBc

FIG. 6B shows the analysis of serology testing for anti-HBc for diagnostic specificity. The x-axis represents the centrifugation time (minutes) and the y-axis the centrifugation speed. The pre-analytical conditions were evaluated as successful (pass) if at least 95/100 (95%) tests achieved a positive test result. The pre-analytical conditions were regarded as not successful if less than 95/100 (95%) tests achieved a positive test result.

Diagnostic Sensitivity Anti-HCV

FIG. 7A shows the analysis of serology testing for anti-HCV for diagnostic sensitivity. The x-axis represents the centrifugation time (minutes) and the y-axis the centrifugation speed. The pre-analytical conditions were evaluated as successful (pass) if at least 9/10 (90%) tests achieved a positive test result. The pre-analytical conditions were regarded as not successful if less than 9/10 (90%) tests achieved a positive test result.

Diagnostic Specificity Anti-HCV

FIG. 7B shows the analysis of serology testing for anti-HCV for diagnostic specificity. The x-axis represents the centrifugation time (minutes) and the y-axis the centrifugation speed. The pre-analytical conditions were evaluated as successful (pass) if at least 95/100 (95%) tests achieved a positive test result. The pre-analytical conditions were regarded as not successful if less than 95/100 (95%) tests achieved a positive test result.

Diagnostic Sensitivity HIV Duo

FIG. 8A shows the analysis of serology testing for HIV duo for diagnostic sensitivity. The x-axis represents the centrifugation time (minutes) and the y-axis the centrifugation speed. The pre-analytical conditions were regarded as successful (pass) if at least 9/10 (90%) tests achieved a positive test result. The pre-analytical conditions were regarded as not successful if less than 9/10 (90%) tests achieved a positive test result.

Diagnostic Specificity HIV Duo

FIG. 8B shows the analysis of serology testing for HIV duo for diagnostic specificity. The x-axis represents the centrifugation time (minutes) and the y-axis the centrifugation speed. The pre-analytical conditions were regarded as successful (pass) if at least 95/100 (95%) tests achieved a positive test result. The pre-analytical conditions were regarded as not successful if less than 95/100 (95%) tests achieved a positive test result.

3. Matrix EDTA Plasma Samples, Blood Grouping Detection

Blood Grouping

FIG. 9 shows the analysis of serology testing for blood grouping. The x-axis represents the centrifugation time (minutes) and the y-axis the centrifugation speed. The pre-analytical conditions were regarded as successful (pass) if data were comparable to the blood typing data under the current routine conditions.

4. Matrix EDTA Plasma Samples, Clinical Chemistry Detection

IgG

FIG. 10 shows the analysis of clinical chemistry testing for IgG. The x-axis represents the centrifugation time (minutes) and the y-axis the centrifugation speed. The pre-analytical conditions were regarded as successful (pass) if data were comparable to the clinical chemistry data under the current routine conditions.

Total Protein

FIG. 11 shows the analysis of clinical chemistry testing for total protein. The x-axis represents the centrifugation time (minutes) and the y-axis the centrifugation speed. The pre-analytical conditions were regarded as successful (pass) if data were comparable to the clinical chemistry data under the current routine conditions.

5. Matrix Citrate Plasma Samples, NAT Detection

Diagnostic Sensitivity HBV

FIG. 12A shows the analysis of NAT testing for HBV for diagnostic sensitivity. The x-axis represents the centrifugation time (minutes) and the y-axis the centrifugation speed. The pre-analytical conditions were regarded as successful (pass) if at least 9/10 (90%) tests achieved a positive test result. The pre-analytical conditions were regarded as not successful if less than 9/10 (90%) tests achieved a positive test result.

Diagnostic specificity HBV FIG. 12B shows the analysis of NAT testing for HBV for diagnostic specificity. The x-axis represents the centrifugation time (minutes) and the y-axis the centrifugation speed. The pre-analytical conditions were regarded as successful (pass) if at least 95/100 (95%) tests achieved a positive test result. The pre-analytical conditions were regarded as not successful if less than 95/100 (95%) tests achieved a positive test result.

Diagnostic Sensitivity HCV

FIG. 13A shows the analysis of NAT testing for HCV for diagnostic sensitivity. The x-axis represents the centrifugation time (minutes) and the y-axis the centrifugation speed. The pre-analytical conditions were regarded as successful (pass) if at least 9/10 (90%) tests achieved a positive test result. The pre-analytical conditions were regarded as not successful if less than 9/10 (90%) tests achieved a positive test result.

Diagnostic Specificity HCV

FIG. 13B shows the analysis of NAT testing for HCV for diagnostic specificity. The x-axis represents the centrifugation time (minutes) and the y-axis the centrifugation speed. The pre-analytical conditions were regarded as successful (pass) if at least 95/100 (95%) tests achieved a positive test result. The pre-analytical conditions were regarded as not successful if less than 95/100 (95%) tests achieved a positive test result.

Diagnostic Sensitivity HIV

FIG. 14A shows the analysis of NAT testing for HIV for diagnostic sensitivity. The x-axis represents the centrifugation time (minutes) and the y-axis the centrifugation speed. The pre-analytical conditions were regarded as successful (pass) if at least 9/10 (90%) tests achieved a positive test result. The pre-analytical conditions were regarded as not successful if less than 9/10 (90%) tests achieved a positive test result.

Diagnostic Specificity HIV

FIG. 14B shows the analysis of NAT testing for HIV for diagnostic specificity. The x-axis represents the centrifugation time (minutes) and the y-axis the centrifugation speed. The pre-analytical conditions were regarded as successful (pass) if at least 95/100 (95%) tests achieved a positive test result. The pre-analytical conditions were regarded as not successful if less than 95/100 (95%) tests achieved a positive test result.

6. Matrix Citrate Plasma Samples, Serology Detection

Diagnostic Sensitivity HBsAG

FIG. 15A shows the analysis of serology testing for HBsAg for diagnostic sensitivity. The x-axis represents the centrifugation time (minutes) and the y-axis the centrifugation speed. The pre-analytical conditions were regarded as successful (pass) if at least 9/10 (90%) tests achieved a positive test result. The pre-analytical conditions were regarded as not successful if less than 9/10 (90%) tests achieved a positive test result.

Diagnostic Specificity HBsAg

FIG. 15B shows the analysis of serology testing for HBV for diagnostic specificity. The x-axis represents the centrifugation time (minutes) and the y-axis the centrifugation speed. The pre-analytical conditions were regarded as successful (pass) if at least 95/100 (95%) tests achieved a positive test result. The pre-analytical conditions were regarded as not successful if less than 95/100 (95%) tests achieved a positive test result.

Diagnostic Sensitivity Anti-HBc

FIG. 16A shows the analysis of serology testing for anti-HBc for diagnostic sensitivity. The x-axis represents the centrifugation time (minutes) and the y-axis the centrifugation speed. The pre-analytical conditions were regarded as successful (pass) if at least 9/10 (90%) tests achieved a positive test result. The pre-analytical conditions were regarded as not successful if less than 9/10 (90%) tests achieved a positive test result.

Diagnostic Specificity Anti-HBc

FIG. 16B shows the analysis of serology testing for anti-HBc for diagnostic specificity. The x-axis represents the centrifugation time (minutes) and the y-axis the centrifugation speed. The pre-analytical conditions were regarded as successful (pass) if at least 95/100 (95%) tests achieved a positive test result. The pre-analytical conditions were regarded as not successful if less than 95/100 (95%) tests achieved a positive test result.

Diagnostic Sensitivity Anti-HCV

FIG. 17A shows the analysis of serology testing for anti-HCV for diagnostic sensitivity. The x-axis represents the centrifugation time (minutes) and the y-axis the centrifugation speed. The pre-analytical conditions were regarded as successful (pass) if at least 9/10 (90%) tests achieved a positive test result. The pre-analytical conditions were regarded as not successful if less than 9/10 (90%) tests achieved a positive test result.

Diagnostic Specificity Anti-HCV

FIG. 17B shows the analysis of serology testing for anti-HCV for diagnostic specificity. The x-axis represents the centrifugation time (minutes) and the y-axis the centrifugation speed. The pre-analytical conditions were regarded as successful (pass) if at least 95/100 (95%) tests achieved a positive test result. The pre-analytical conditions were regarded as not successful if less than 95/100 (95%) tests achieved a positive test result.

Diagnostic Sensitivity HIV Duo

FIG. 18A shows the analysis of serology testing for HIV duo for diagnostic sensitivity. The x-axis represents the centrifugation time (minutes) and the y-axis the centrifugation speed. The pre-analytical conditions were regarded as successful (pass) if at least 9/10 (90%) tests achieved a positive test result. The pre-analytical conditions were regarded as not successful if less than 9/10 (90%) tests achieved a positive test result.

Diagnostic Specificity HIV Duo

FIG. 18B shows the analysis of serology testing for HIV duo for diagnostic specificity. The x-axis represents the centrifugation time (minutes) and the y-axis the centrifugation speed. The pre-analytical conditions were regarded as successful (pass) if at least 95/100 (95%) tests achieved a positive test result. The pre-analytical conditions were regarded as not successful if less than 95/100 (95%) tests achieved a positive test result.

7. Matrix Citrate Plasma Samples, Blood Grouping Detection

FIG. 19 shows the analysis of serology testing for blood grouping. The x-axis represents the centrifugation time (minutes) and the y-axis the centrifugation speed. The pre-analytical conditions were regarded as successful (pass) if data were comparable to the blood typing data under the current routine conditions.

8. Matrix Citrate Plasma Samples, Clinical Chemistry Detection

IgG

FIG. 20 shows the analysis of clinical chemistry testing for IgG. The x-axis represents the centrifugation time (minutes) and the y-axis the centrifugation speed. The pre-analytical conditions were regarded as successful (pass) if data were comparable to the clinical chemistry data under the current routine conditions.

Total Protein

FIG. 21 shows the analysis of clinical chemistry testing for total protein. The x-axis represents the centrifugation time (minutes) and the y-axis the centrifugation speed. The pre-analytical conditions were regarded as successful (pass) if data were comparable to the clinical chemistry data under the current routine conditions.

1. A method for the pre-analytical treatment of a blood sample to be analyzed, comprising the following steps:
a) providing the sample to be analyzed in a suitable container,
b) providing a suitable sample matrix for said sample, comprising an anticoagulant selected from K₂EDTA, K₃EDTA and sodium citrate in said container,
c) centrifuging said sample of b) at about 2000 to 3400×g for about 2 to 10 min; and
d) subjecting said sample to analytical testing comprising at least two of i) serology testing, ii) clinical chemistry (CC) testing, iii) nucleic acid testing (NAT); and iv) blood typing.

2. The method according to claim 1, further comprising archiving analytical material of i) and/or iii).

3. The method according to claim 1, wherein said container is labeled with a barcode.

4. The method according to claim 1, wherein said sample has a volume of about 5 to 10 ml.

5. The method according to claim 1, wherein said samples for analytical testing are selected from about 900 ul for serology, about 100 ul for CC, about 2 ml for NAT, and about 200 ul for blood typing.

6. The method according to claim 1, wherein said container is a sample tube, vial, or round bottom tube.

7. The method according to claim 1, wherein said sample matrix is provided as a solution and/or a spray dried composition.

8. The method according to claim 1, wherein said method is performed fully automated.

9. The method according to claim 1, wherein said blood sample to be analyzed is not a serum sample and/or does not comprise heparin.

10. The method according to claim 1, wherein said blood sample to be analyzed is a pooled sample.

11. The method according to claim 1, further comprising an additional centrifugation at about 2000 to 3400×g for about 15 to 25 min and a re-testing of serology according to claim 1 d) i), if the sample is initially reactive for said serology.

12. An apparatus, characterized in that it comprises suitable components for performing the method according to claim 1.

13. The apparatus according to claim 12, characterized in that it is suitable for generating the sample matrix, centrifugation, aliquot extraction, serology testing, clinical chemistry testing, nucleic acid extraction including, pooling, PCR preparation, blood typing, and/or raw data analysis.

14. The apparatus according to claim 12, characterized in that all components are designed as an integrated apparatus and are located within a housing.

15. A method for the pre-analytical treatment of a blood sample to be analyzed, comprising the following steps:
a) providing the sample to be analyzed in a suitable container,
b) providing a suitable sample matrix for said sample, comprising an anticoagulant selected from K₂EDTA, K₃EDTA and sodium citrate in said container,
c) centrifuging said sample of b) at about 2000 to 3400×g for about 2 to 10 min; and
d) subjecting said sample to analytical testing comprising at least two of i) serology testing, ii) clinical chemistry (CC) testing, iii) nucleic acid testing (NAT); and iv) blood typing;
wherein said method comprises the use of an apparatus according to claim 12.

16. The method, according to claim 1, wherein, in step c) the sample is centrifuged for about 5 min at 2600×g.

17. The method, according to claim 1, comprising, in step d) subjecting said sample to analytical testing comprising i) serology testing, ii) clinical chemistry (CC) testing, iii) nucleic acid testing (NAT); and iv) blood typing.

18. The method, according to claim 4, wherein said sample has a volume of about 9 ml.

19. The method, according to claim 10, wherein 2 to 15 samples are pooled.

20. The method, according to claim 11, wherein the additional centrifugation is carried out for about 20 min at about 2600×g.
